(19) United States
(12) Patent Application Publication
(10) Pub. No.: US 2006/0106098 A1
(43) Pub. Date: May 18, 2006

(54) Andrographolide and its derivatives as TNF-alpha antagonists

(75) Inventors: Li-Wei Hsu, Taichung City (TW); Su-Chen Chang, Taichung City (TW); Chen-Hsiang Shen, Taipei County (TW); Kuo-Sheng Chuang, Hualien County (TW)

Correspondence Address: Scully, Scott, Murphy & Presser, 400 Garden City Plaza, Garden City, NY, 11530-3319, US

(73) Assignee: Advanced Gene Technology, Corp., Taichung City (TW)

(21) Appl. No.: 10/992,180

(22) Filed: Nov. 18, 2004

(51) Int. Cl. C07D 307/32; A61K 31/365

(52) U.S. Cl. 514/473; 549/313

(57) ABSTRACT

The present invention relates to andrographolide and its derivatives of the general formula (I), as well as the stereoisomers and salts of andrographolide and the derivatives. 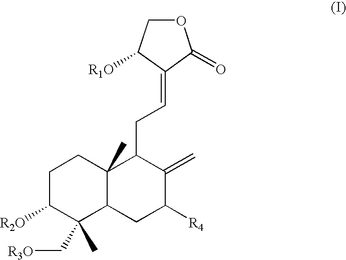
Andrographolide and its derivatives represented by general formula (I) defined above are useful as TNFα (tumor necrosis factor alpha) antagonists or inhibitors which have inhibitory effect on the binding of TNFα to TNF-RI. Andrographolide exhibited inhibitor activity with IC₅₀ values 60 μM on L929 cell proliferation/cytotoxicity assay without cell cytotoxicity. In addition, in the animal model test of collagen-induced arthritis, andrographolide exhibited 50% paw edema. Andrographolide and its derivatives are promising sources with high TNFα-inhibiting or antagonizing activity.

### FIELD OF THE INVENTION

The present invention to a novel pharmaceutical composition comprising andrographolide and/or its derivatives and useful as a TNFα antagonist or inhibitor. The present invention also relates to novel derivatives of andrographolide.

### BACKGROUND OF THE INVENTION

Andrographolide has the formula (I),

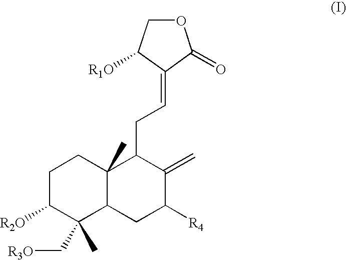

wherein R₁═R₂═R₃═R₄═H. It is the major constituent of the plant Andrographis paniculata, and was first isolated by Gorter¹. Andrographolide, classified as a diterpene lactone, has been shown to possess hepatoprotective², immunostimulant³, anti-inflammatory⁴, antioxidant⁵, anticancer⁶, anti-HIV infection⁷, anti-bacterial⁸, antipyretic, antiinfective⁹, analgesic¹⁰, anti-thrombotic¹¹, anti-cardiovascular¹², anti-hypersensitive¹³, anti-allergic¹⁴, anti-diarrheal¹⁵ and anti-hyperglycemic¹⁶ activities. In anti-inflammatory activity, andrographolide was reported to have a synergistic inhibition of cyclooxygenase-2¹⁷, suppression of rat neutrophil reactive oxygen species production and adhesion^{4b}, suppression of inducible nitric oxide synthase expression^{4d}, and inhibition of the TNFα-induced upregulation of ICAM-1 expression and endothelial-monocyte adhesion¹⁸. However the anti-inflammatory mechanism has not yet been established. Of the several inflammatory mediators known to date, TNFα is one of by far the most potent and characterized cytokines, it is selected to test whether andrographolide inhibits the binding of TNFα to TNFα-RI by L929 cell proliferation/cytotoxicity assay.

TNFα (tumor necrosis factor-α) plays an important role in the host defense. It cause resistance to many pathogenic microorganisms and some viruses. Even if TNFα has undoubtedly a beneficial function (mainly on the systematic level), it could lead to pathological consequences. TNFα plays a significant role in the pathogenesis of septic shock, characterized by hypotension and multiple organ failure among others. TNFα is the main mediator of cachexia characterized by abnormal weight-loss of cancer patients. Often TNFα is detected in the synovial fluid of patients suffering from arthritis. There was a broad spectrum of diseases, where TNFα could play an important role. Compounds binding with TNFα receptor may be therefore useful in the treatment of numerous diseases or conditions in which TNFα is involved, such as rheumatoid arthritis, Crohn's disease, plaque sclerosis, septic shock, cancer or cachexia associated with an immunodeficiency.

### SUMMARY OF THE INVENTION

The present invention relates to novel TNFα antagonists or inhibitors, their stereoisomers, their pharmaceutical acceptable salts. The present invention particularly relates to novel derivatives of andrographolide, its stereoisomers and salts. The novel derivatives of andrographolide have the general formula (I):

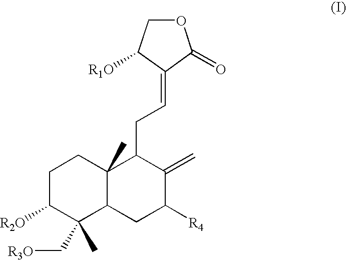

where R¹, R² and R³ may be same or different and independently represent substituted or unsubstituted groups such as alkyl, aryl, heteroaryl, aralkyl, heteroaralkyl, alkanoyl, alkenoyl, aroyl, heteroaroyl, aralkanoyl, arakenoyl, heteroaralkanoyl, heteroaralkenoyl, sulfonyl group or a group —(CO)—W—R⁵ where W represents O, S or NR⁶, wherein R⁶ represents hydrogen or (C₁-C₆)alkyl group, R⁵ represents substituted or unsubstituted groups such as alkyl, aryl, aralkyl, aroyl, OR², OR³ or a substituted or unsubstituted 6 or 7 membered cyclic structure containing carbon and oxygen atoms; R⁴ represents halogen or XR⁷ where X represents O, S, NH and R⁷ represents hydrogen or substituted or unsubstituted groups such as alkyl, aryl, aralkyl, alkenoyl, alkanoyl, aroyl, heteroaroyl, aralkenoyl, aralkanoyl, sulfonyl groups or a group —(CO)—NH—R⁸ where R⁸ represents substituted or unsubstituted groups such as alkyl, aryl and aralkyl. Andrographolide is a compound of the abovesaid formula (I) where R₁═R₂═R₃═R₄═H.

Andrographolide and its derivatives represented by general formula (I) defined above of the present invention are useful for inhibiting the release of TNFα and therefore may be used in the treatment of numerous pathologies in which TNFα is involved, such as rheumatoid arthritis, Crohn's disease, plaque sclerosis, septic shock, cancer or cachexia associated with an immunodeficiency.

The present invention also relates to a pharmaceutical composition for inhibiting or antagonizing TNFα in a mammal, including human, comprising an amount of a compound of formula (I), or a pharmaceutically acceptable salt thereof, that is effective in inhibiting or antagonizing TNFα, and a pharmaceutically acceptable carrier.

The present invention also relates to a pharmaceutical composition for treating diseases or conditions in which TNFα is involved, such as rheumatoid arthritis, Crohn's disease, plaque sclerosis, septic shock, cancer or cachexia associated with an immunodeficiency, comprising an amount of a compound of formula (I), or a pharmaceutically acceptable salt thereof, that is effective in inhibiting or antagonizing TNFα, and a pharmaceutically acceptable carrier.

The present invention also relates to a method of inhibiting or antagonizing TNFα in a mammal, including human, comprising administering to said mammal an amount of a compound of formula (I), or a pharmaceutically acceptable salt thereof, that is effective in inhibiting or antagonizing TNFα.

The present invention also relates to a method of treating diseases or conditions in which TNFα is involved, such as rheumatoid arthritis, Crohn's disease, plaque sclerosis, septic shock, cancer or cachexia associated with an immunodeficiency, comprising an amount of a compound of formula (I), or a pharmaceutically acceptable salt thereof, that is effective in treating such diseases or conditions.

### BRIEF DESCRIPTION OF THE DRAWINGS

The accompanying drawings are to provide a further understanding of the invention, and are incorporated in and constitute a part of this specification. The drawings illustrate embodiments of the invention and, together with the description, serve to explain the principles of the invention.

FIG. 1 shows SDS-PAGE (Sodium Dodecyl Sulfate Polyacrylamide Gel Electrophoresis) of purified rhTNFα (recombinant human TNFα) (lane 2) and biotinylated rhTNFα (lane 3).

FIG. 2 is a chromatogram of the crude methanolic extract of Andrographis paniculata Nees on a TSK Gel ODS 80™ (TOSOH) reverse phase column (250×4.6 mm) filled with of the 5 μm gel particle.

FIG. 3 is a chromatogram of the crude water extract of Andrographis paniculata Nees on a TSK Gel ODS 80™ (TOSOH) reverse phase column (250×4.6 mm) filled with of the 5 μm gel particle.

FIG. 4 is a chromatogram of andrographolide on a TSK Gel ODS 80™ (TOSOH) reverse phase column (250×4.6 mm) filled with of the 5 μm gel particle.

FIG. 5 shows the inhibitory and cytotocixity percentage of andrographolide aqueous solution on in vitro L929 cell proliferation/cytotoxicity assay on microtiter plates.

FIG. 6A shows a hind paw of a rat with collagen-induced arthritis. Swelling and erythema were observed.

FIG. 6B shows hind paw of a rat before collagen induction.

FIG. 7A. shows volume changes in decreased percentage of left hind paw for the group treated with andrographolide. The volume of T0 is before injection with CII-INF, T1 is before treatment, T3 is the 6^{th} day of treatment, T4 and T5 are the 3^{rd} day and the 7^{th} day 7th after administration of andrographolide.

FIG. 7B. Edema percentage of paws, calculated by volume changes, in comparison of treatment and non-treatment at various time points. T3 is 1-(T3-T1/T1-T0)%, T4 is 1-(T1-T4/T1-T0)% and T5 is 1-(T1-T5/T1-T0)%.

FIG. 8A. shows volumes of left hind paw for the group treated with dexamethasone. The volume of T0 is before injection with CII-INF, T1 is before treatment, T3 is the 6^{th} day of treatment, T4 and T5 are the 3^{rd} day and the 7^{th} day after administration.

FIG. 8B. Edema percentage of paws, calculated by volume changes, in comparison of treatment and non-treatment at various time points. T3 is 1-(T3-T1/T1-T0)%, T4 is 1-(T1-T4/T1-T0)% and T5 is 1-(T1-T5/T1-T0)%.

FIG. 9 shows a normal histological slice of joint of non-immune with collagen II.

FIG. 10 shows a histopathological slice of a rat with CIA and treated (IP) with andrographolide. Proliferation of synovial lining cell and infiltration of erythrocytes and some lymphocytes were observed.

FIG. 11 shows a histopathological slice of a rat with CIA and treated (IP) with andrographolide by oral. Proliferation of synovial lining cell and infiltration of lymphocytes were found.

FIG. 12 shows a histopathological slice of a rat with CIA and treated (IP) with andrographolide (by external application). Proliferation of synovial lining cell and loosely connective tissues appeared.

FIG. 13 shows a histopathological slice of a rat with CIA and treated (IP) with dexamethasone. Proliferation of synovial lining cell and infiltration of erythrocytes and some lymphocytes were observed.

FIG. 14 shows a histopathological slice of a rat with CIA and treated (IP) with 5% ethanol, Proliferation of synovial lining cell and infiltration of lymphocytes were observed.

FIG. 15 shows a histopathological slice of a rat with CIA and treated (IP) with dexamethasone. Periarticular edema and infiltration of lymphocytes were found.

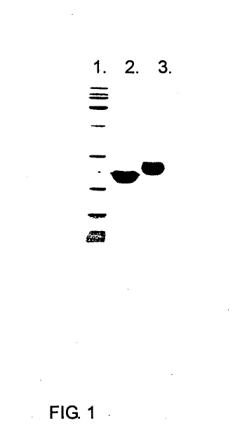

### DETAILED DESCRIPTION OF THE INVENTION AND PREFERRED EMBODIMENTS

The compound of formula (I) or a pharmaceutically acceptable salt thereof according to the present invention may be administered to mammals via either the oral, parenteral (such as subcutaneous, intravenous, intramuscular, intrasternal and infusion techniques), rectal, intranasal, topical or transdermal (e.g., through the use of a patch) routes. The compound of formula (I) or a pharmaceutically acceptable salt thereof may be administered alone or in combination with pharmaceutically acceptable carriers or diluents by either of the routes previously indicated, and such administration may be carried out in single or multiple doses. Suitable pharmaceutical carriers include solid diluents or fillers, sterile aqueous media and various non-toxic organic solvents, etc.

### EXPERIMENTAL PROCEDURES

1. Purification of rhTNFα and Biotinylation rhTNFα from E. coli.

Expression of rhTNFα (recombinant human TNFα) protein in E. coli BL21 (DE3) had been induced by IPTG (isopropyl-beta-D-thiogalactoside) and purified by different concentration of imidazole under native condition.

2. Preparation of the Methanolic Extract of A. Paniculata

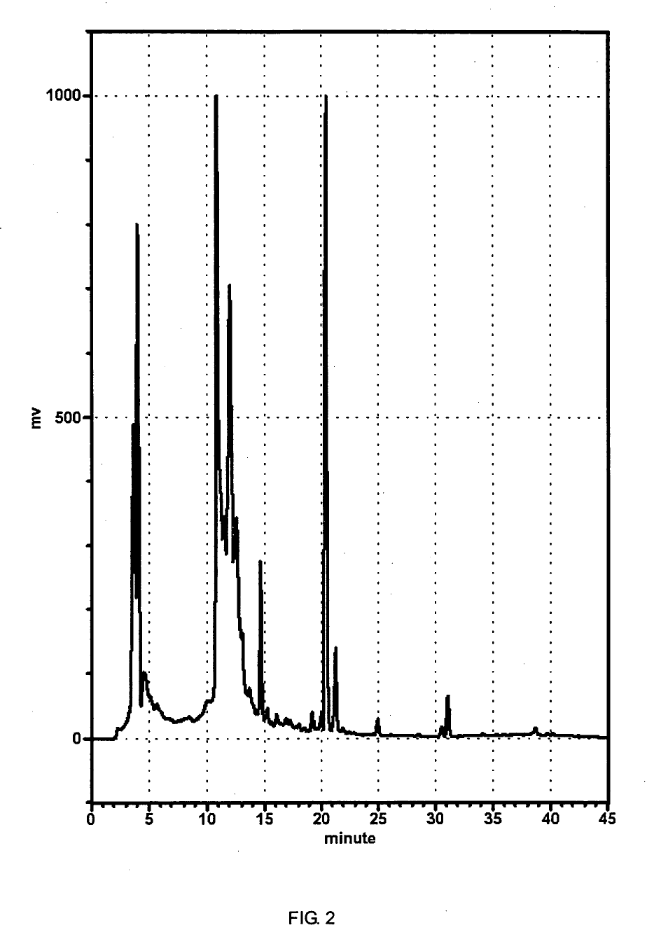

Fifty grams of A. paniculata was washed and dried, then methanol was added to the weighted herb (40/1, v/w) to extract the herbal ingredients for 3 days at room temperature. The extract was filtered and the filtrate was concentrated under rotatory evaporator (Heidolph Laborota 4000) until the final volume was reduced to about 50 ml. FIG. 2 shows the chromatogram of the crude methanolic extract of Andrographis paniculata Nees on a TSK Gel ODS 80™ (TOSOH) reverse phase column (250×4.6 mm) filled with of the 5 μm gel particle.

3. Preparation of the Water Extract of A. Paniculata

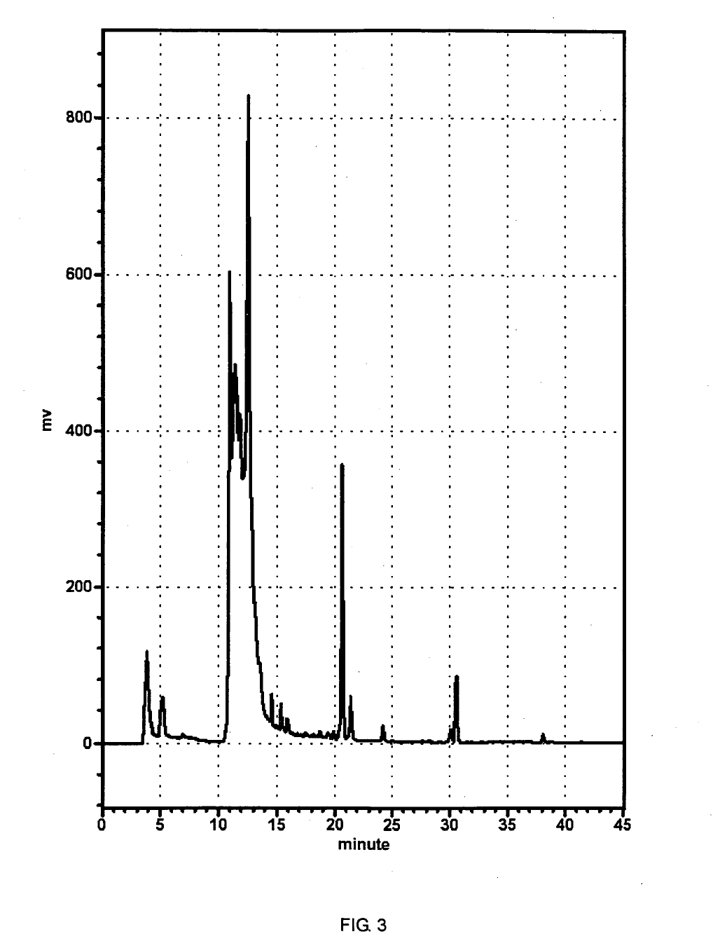

Fifty grams of A. paniculata was washed and dried, and then water was added to the weighted herb (10/1, v/w) to extract the herbal ingredients twice for 2 hours at 95° C. The extract was filtered and the filtrate was concentrated under rotatory evaporator (Heidolph Laborota 4000) until the final volume was reduced to about 100 ml. FIG. 3 shows the chromatogram of the crude water extract of Andrographis paniculata Nees on a TSK Gel ODS 80™ (TOSOH) reverse phase column (250×4.6 mm) filled with of the 5 μm gel particle.

4. Purification of Andrographolide

Fifty grams of A. paniculata leaf was extracted twice with 95% absolute ethanol 2 hours at boiling temperature. The extract was filtered and the solvent was removed from the filtrate under vacuum. The dark green crystalline obtained was washed separately with toluene several times until most of the coloring matter was removed from the residue. Then the toluene was completely removed from the residue. The crystalline material left behind was dissolved in hot methanol and cool in a refrigerator for crystallization. The process was repeated several times until colorless crystals were obtained.

5. Andrographolide Identification

(1) Thin-Layer Chromatography

For TLC experiment, pre-coated plates of silica gel 60F₂₅₄(E. Merck) were used and spotting was done on capillary tube. The plates were scanned on a UV observed box (Gamag). The solvent system was chloroform: ethanol (9:1) for pure andrographolide. TLC of the isolated andrographolide showed a single spot with its R_{f} value 0.46 in this solvent system.

(2) UV/Vis is Absorption Spectrum

UV absorption spectrum of andrographolide and reference standard in methanol were recorded on UV/VIS spectrophotometer (Beckman). The maximum absorption band (λₘₐₓ) of andrographolide in H₂O show λₘₐₓ=230 nm with absorption coefficient 13000 and in methanol show λₘₐₓ=222 nm with absorption coefficient 13200.

(3) LC/MS Spectrum

The atmospheric pressure ionization with ESI mass spectrum of molecular ions was obtained on a LC/MS (liquid chromatography-mass spectrometry) (Varian). The mobile phase was MeOH+2 mM NH₄OAc. Andrographolide Mass: 351 (M+H)⁺, 368 (M+NH₄)⁺, 373 (M+Na)⁺.

(4) HPLC Spectrum

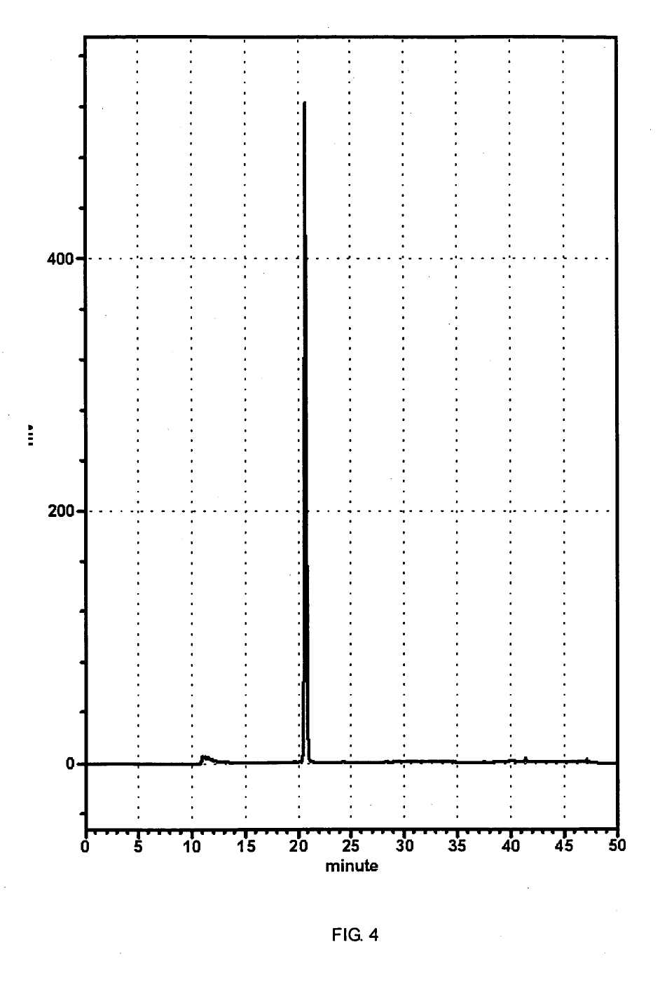

The HPLC spectra of andrographolide and reference standard were obtained by TSK Gel ODS 80™ (5 μm) TOSOH reverse phase column (4.6×250 mm) using a Shimadzu HPLC system with mobile phase containing acetonitrile and water (1:1) under 0˜100 gradient at flow rate of 0.75 ml/min and the eluted compound detected at 230 nm. The mobile phase used was a mixture of acetonitrile (B) and H₂O (A) at a flow rate of 0.75 ml/min. The column was sequentially eluted as follows: 0%˜30% B for the first 5 minutes; a linear gradient of 30˜50% B for 5 to 25 minutes; 50˜70% B for 25 to 30 minutes; 70˜1 00% B for 30 to 35 minutes; 100% B for 35 to 45 minutes. The detection was performed at a wavelength of 230 nm with a detection sensitivity of 0.01 AUFS. The HPLC analysis of the andrographolide gave a single peak with retention time of 20.5 min as with the standard andrographolide. FIG. 4 shows the chromatogram of andrographolide on a TSK Gel ODS 80™ (TOSOH) reverse phase column (250×4.6 mm) filled with of the 5 μm gel particle.

6. Inhibitory Effect of Andrographolide on TNFα Binding to TNFα-RI

Different ratio of andrographolide and biotinylated rhTNF-α mixture was pre-incubated at 37° C. for 30 min. The mixture was then added into a 96-well microtiter plate which was pre-coated with rhTNF-α receptor (rhTNF-αR1). After 2 hr-incubation at 37° C., the plate was washed with TBST (Tris-buffered saline/Tween 20). Alkaline phosphatase-conjugated avidin was used for detection. The density of wavelength at 405 nm was measured, and the inhibition ability of andrographolide was evaluated as the following:
Inhibition (%)=100−O.D. andrographolide/O.D. PC(positive control) %.
7. TNFα Antagonist Activity Evaluation by In Vitro L929 Cell Proliferation/Cytotoxicity Assay

In vitro L929 cell proliferation/cytotoxicity assay was performed on microtiter plates. L929 cells were cultured in Eagle's Minimal Essential Medium (EMEM) containing 10% bovine serum, 1% P/S and 1% non-essential amino acid. Confluent L929 cells were washed with 2 ml PBS solution and then trypsinized, followed by resuspending in complete medium. Two hundred μl cell suspension was aspirated for counting the cell density and the other suspension was centrifuged at 1500 rpm for 5 min. The supernatant was removed and the complete medium was added to dilute cells to a final concentration of 1.5×10⁵ cells/ml. One hundred μl of cell suspension was added to each well in 96-well flat-bottomed microtiter plates and incubated for 24 hrs in 5% CO₂ atmosphere at 37° C.

The chemical was resuspended in 1×PBS and sterilized with 0.22 μm filters. Various concentrations of chemical were incubated for 1 hr with equal volume of commercial TNFα0.2 ng/ml. Prior to the end of the 1 hr pre-incubation, the medium was removed from the 24 hr incubated 96-well plate into which 50 μl fresh medium with 4 μg/ml of Actinomycin D was added. The 50 μl of pre-incubated mixture of chemical and TNFα was transferred to the 96-well plate with the medium containing Act D to give the final concentration of Act D 2 μg/ml, TNFα0.1 ng/ml. Act D 2 μg/ml and TNFα0.1 ng/ml were added as positive control and Act D 2 μg/ml only was added as a negative control. After gently shaking to mix, the 96-well plate was incubated for 24 hrs in 5% CO₂ atmosphere at 37° C.

Cytotoxicity

The same samples as those for TNFα activity assay were added to the 96-well plate with the medium containing Act D to give the final concentration of Act D 2 μg/ml. The 96-well plate was gently shaken and incubated for 24 hrs in 5% CO₂ atmosphere at 37° C. 50 μl XTT (2,3-bis(2-methoxy-4-nitro-5-sulfophenyl)-5-[((phenylamino)carbonyl]-2H-tetra -zolium hydroxide) mixture was added to each well, and incubated for 4 hrs. Read with ELISA reader at O.D. 490/630 nm.
Calculation of the TNFα Activity Inhabitation and Cytotoxicity

$$TNF α Inhibition \% = \frac{O . D {.}_{dilut + TNF + Act .} - O . D {.}_{TNF α + Act}}{O . D {.}_{Act only} - O . D {.}_{TNF α + Act}} \times 100 \%$$

$$Cytotoxicity \% = \frac{O . D {.}_{dilut . + ActD}}{O . D {.}_{ActD only}} \times 100 \%$$

8. The Effect of the Methanolic Extract of A. Paniculata on L929 Cell Proliferation/Cytotoxicity Assay

The inhibition effect of methanolic extract of A. Paniculata on TNFα was measured by the same procedure as described previously by in vitro L929 cell proliferation/cytotoxicity assay on microtiter plates. The result showed that methanolic extract of A. Paniculata as a TNFα antagonist with a 40% inhibitory effect at 500 μg/ml methanol extract while no cytotocixity was observed.

9. The Effect of the Water Extract of A. Paniculata on L929 Cell Proliferation/Cytotoxicity Assay

The effect of water extract of A. Paniculata on TNFα inhibitor activity was measured by the same procedure as described above. The result showed that water extract of A. Paniculata with 34% inhibitory percentage at 500 μg/ml water extract as a TNFα antagonist and no cytotocixity was found.

10. Finding Inhibitors of TNFα from Andrographis Paniculata Water Extract by L929 Cell Proliferation/Cytotoxicity Assay

The possible TNFα inhibitor candidates were found from herbal ingredients fractionalized by HPLC from herbal extract. After the collected herbs were washed and dried, water was added to the weighted herb (10/1, v/w) to extract the herbal ingredients. The extraction procedure includes blending the mixture and pooling the supernatant after centrifugation at 8000 rpm for 30 minutes. Repeat the procedure for two times. All the supernatant was collected and concentrated with a rotatory evaporator (Heidolph Laborota 4000) until the final volume was reduced to about 50 ml.

Then a separation procedure was performed. One hundred μl of the concentrated supernatant (i.e. the herb extract) was applied to a pre-equilibrated HPLC system (Shimadzu). A TSK Gel 80™ reverse phase column (TOSOH) was used for separation. The solvent used for separation were double distilled water and absolute ethanol under 0-100% gradient for 96 minutes at a flow rate of 0.75 ml/min.

One-minute fractions were collected and dried using a SpeedVac (Savant). Each fraction was re-dissolved in 1 ml H₂O for screening TNFα inhibitors. The fractions with TNFα inhibitor activity were then further purified by HPLC until the purity was more than 95%.

A compound having TNFα inhibitor activity was found in the methanolic extract of A. paniculata by using the procedures described above. The crude methanolic extract of A. paniculata was fractionalized on a TSK Gel ODS 80™ (TOSOH) reverse phase column. The particle size of the gel in this column was 5 μm, and the column size was 250×4.6 mm. The mobile phase used was a mixture of acetonitrile (B) and H₂O (A) at a flow rate of 0.75 ml/min. The column was sequentially eluted as follows: 0%˜30% B for the first 5 minutes; a linear gradient of 30˜50% B for 5 to 25 minutes; 50˜70% B for 25 to 30 minutes; 70˜100% B for 30 to 35 minutes; 100% B for 35 to 45 minutes. The detection was performed at a wavelength of 230 nm with a detection sensitivity of 0.01 AUFS. These fractions with TNFα inhibitor activity were purified from collected solution with retention 20-21 min a TSK Gel ODS 8™ (TOSOH) reverse phase column. The fractions were characterized by LC/MS and with the same retention time as standard andrographolide by HPLC.

11. Evaluation for Andrographolide as TNFα Antagonist by In Vitro L929 Cell Proliferation/Cytotoxicity Assay

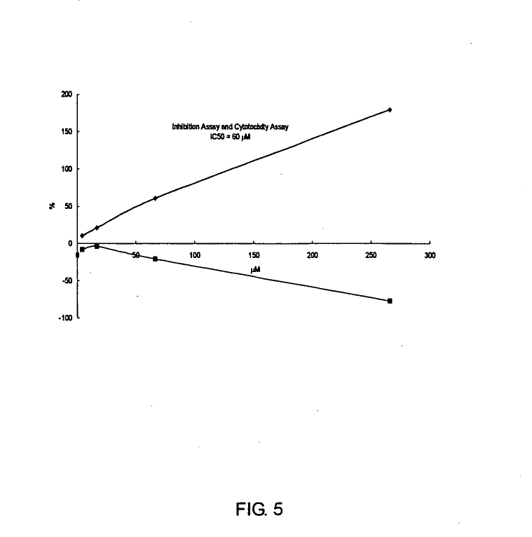

The inhibition effect of andrographolide on TNFα was measured by the same procedure as described previously by in vitro L929 cell proliferation/cytotoxicity assay on microtiter plates. The inhibition and cytotocixity effect of andrographolide on the TNFα inhibitor potential on L929 cell line was shown in FIG. 5. The result in FIG. 5 showed that the inhibition percentage was increased as andrographolide concentration increases, and the IC50 was found to be 60 μM as TNFα antagonist. No cytotocixity was found at this concentration.

12. Anti-Inflammatory Effect of Andrographolide on Rats with Collagen-Induced Arthritis Model

SD rats of SPF grade were supplied from BioLasco. Prior to executing study, the animals were accommodated for 4 days after received. Weighing, blood sampling, measuring the paw volumes and other related records for each animal were established. The rats were immunized and boosted with bovine collagen II-IFA (Incomplete Freund's Adjuvant, from Sigma) to induce arthritis (CIA). The CIA rats were grouped into 6 groups and daily injected with the drug candidate, andrographolide. Dexamethasone (0.2 mg) was used as a positive control and 5% ethanol as a negative control. Treatment period was 7 days. Body weight and paw volumes were measured and blood sampling were collected at day 0, 3, 6, 10 and 14. Six days after the final dosing, all the animals were sacrificed. The affected hind limbs were removed for histological assessment. The parameters of body weights and paw volumes were measured and compared for before, during and after treatment with the drug candidate.

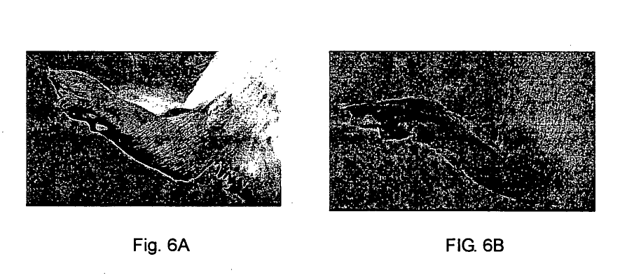

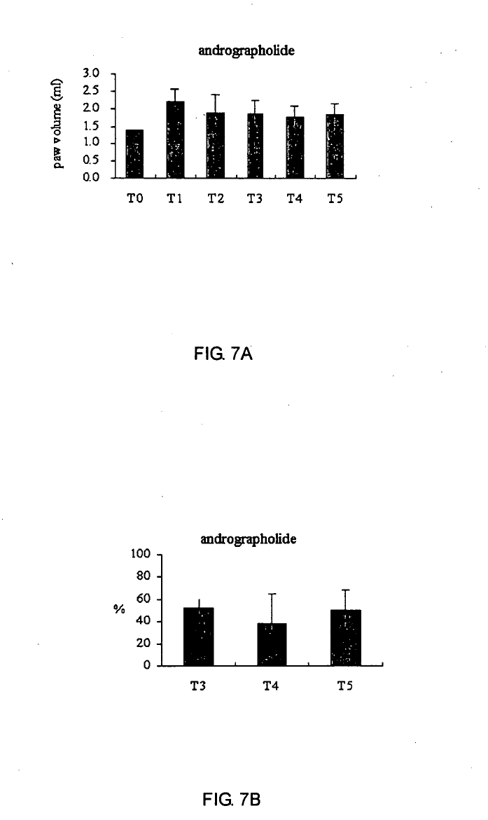

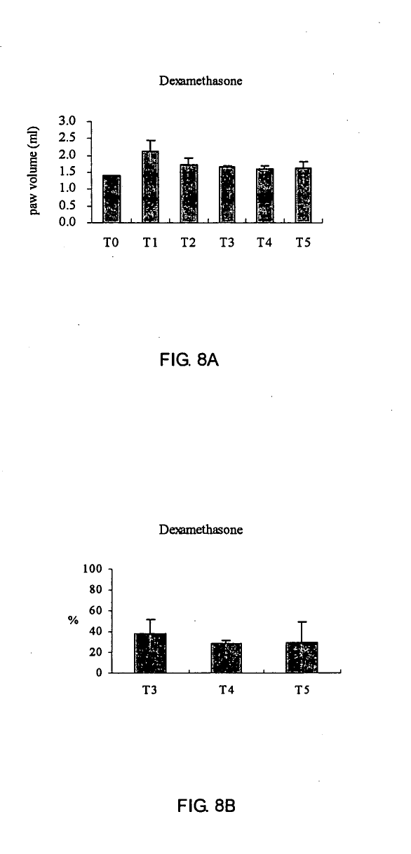

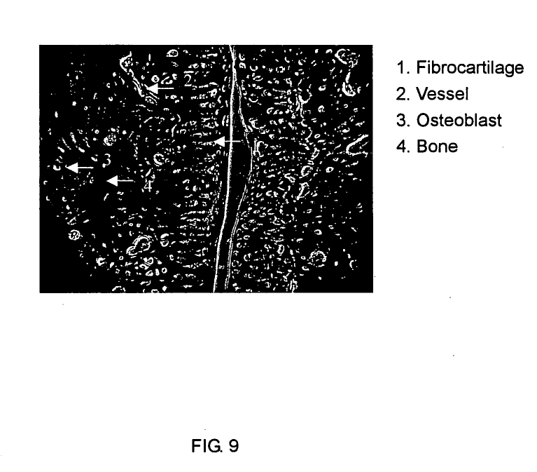

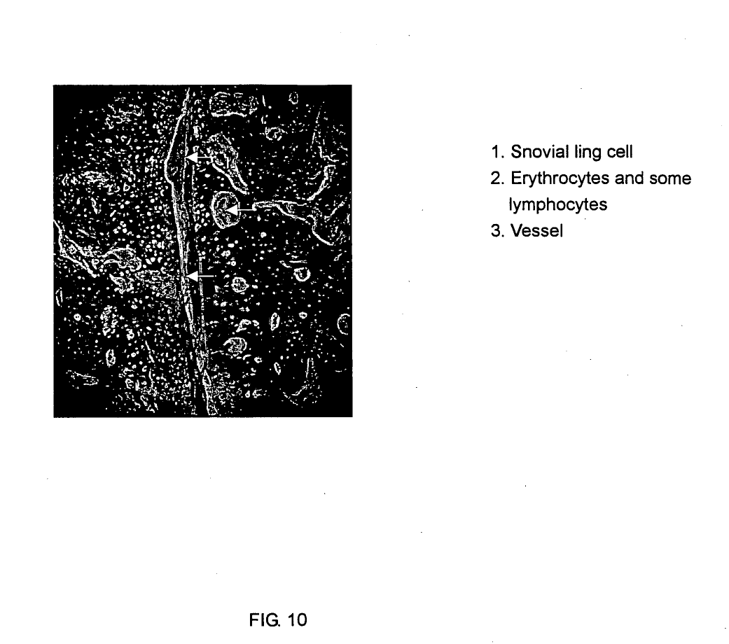

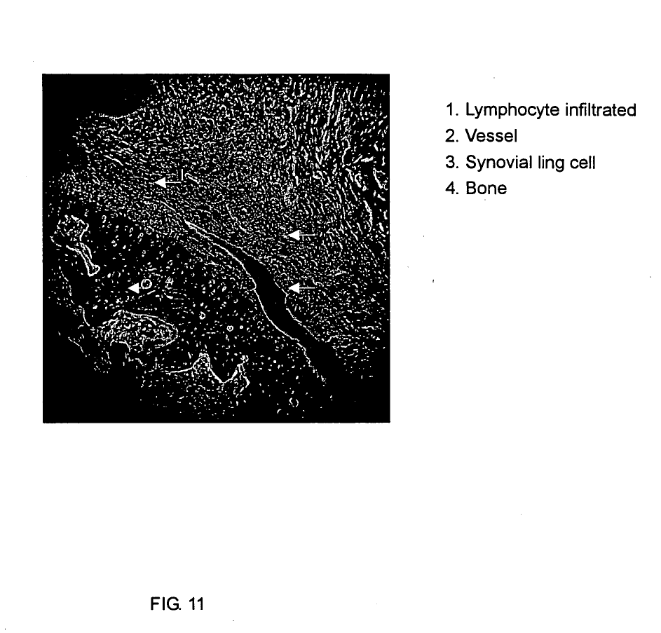

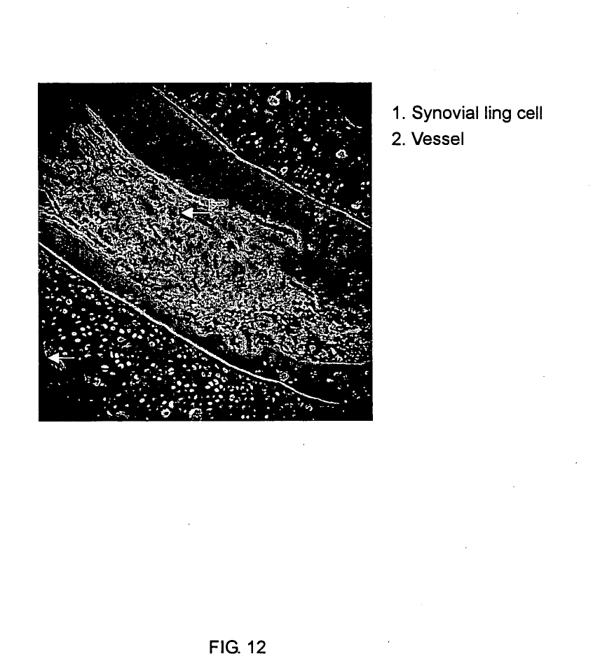

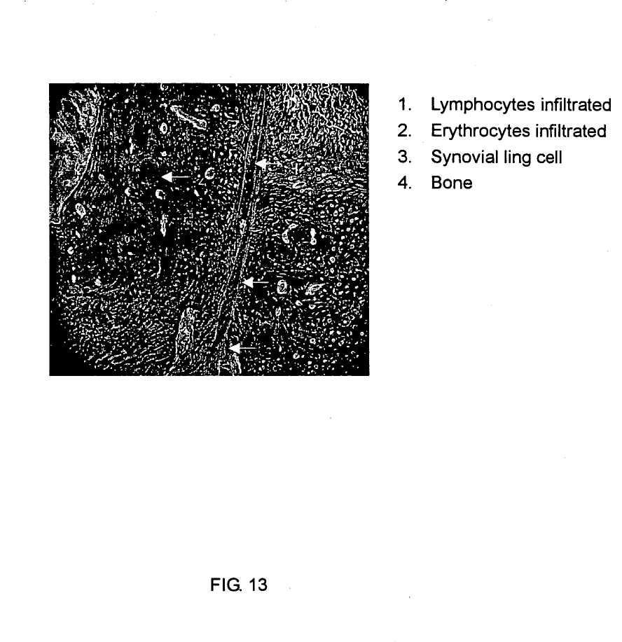

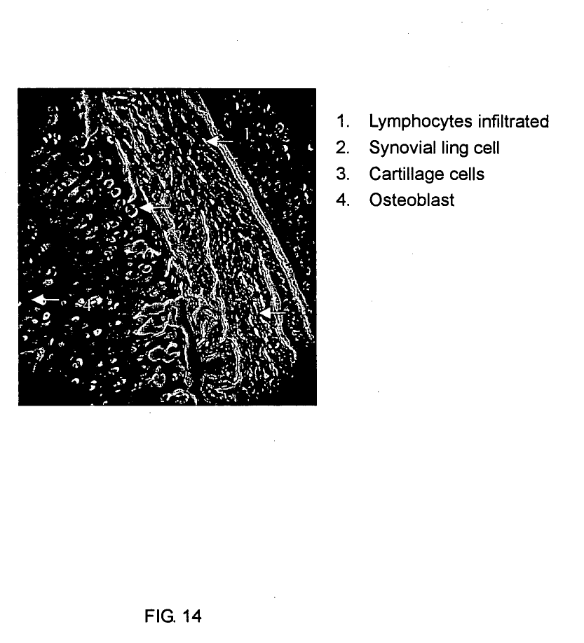

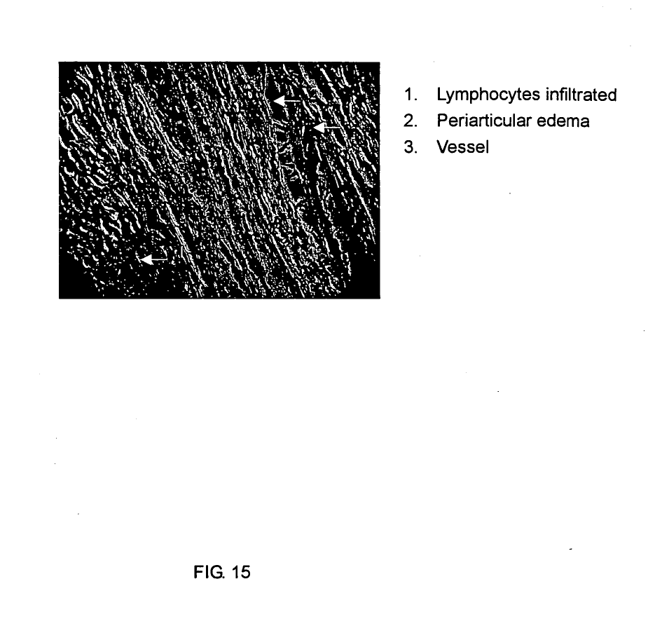

Collagen-induced arthritis was found on day 9th after boosting, the volumes of hind paw swelled to 2-2.5 times that of normal hind paws (FIGS. 6A and 6B). The groups treated with andrographolide appeared decreased percentage, 48.1%, of edema volume of hind paws. The decreased percentage of edema were 61.43% and 50.24% on the 3^{rd} day and the 7^{th} day after treatment stopped (FIGS. 7A and 7B). The Group treated with dexamethasone was used as positive control. The decrease in edema volume during the treatment period in comparison with non-treatment was 28.21% on the 3^{rd} day and 29.97% on the 7^{th} day after administration stopped (FIGS. 8A and 8B). Observable histopathological changes, loose connective tissues, lymphocytes infiltration around joint, periarticular edema and proliferation of synovial lining cell could be observed in all arthritis samples (FIG. 10 to 15) but not in normal samples (FIG. 9)

### SYNTHESIS EXAMPLE

### Preparation of 3-acetoxy andrographolide

A mixture of andrographolide (5 g) and trityl chloride (10 g) in dry pyridine (30 ml) was heated at 60° C. for 6 h. The reaction was monitored by TLC. After completion of the reaction, the reaction mixture was cooled, diluted with diethylether. The organic layer was washed with aqueous copper sulphate solution followed by water and dried over Na₂SO₄. The residue obtained after removal of the solvent was chromatographed over column of silica gel (230-400 mesh, using light petrol: ethyl acetate=6:4 as an eluent) to obtain 19-trityl andrographolide (5 g).

19-Trityl andrographolide (1 g) obtained in the above step was refluxed in distilled acetic anhydride (40 ml) for 5 min. After completion of the reaction (monitored by TLC), the contents were cooled to room temperature, diluted with water and extracted with dichloromethane. The organic layer was separated, dried over Na₂SO₄ and concentrated. The crude material was purified by flash chromatography(silica gel: 230-400 mesh, eluting system chloroform and acetone=95:5) to obtain pure 3-acetoxy-19-trityl andrographolide (300 mg).

3-Acetoxy-19-trityl andrographolide (300 mg) obtained was treated with a mixture of formic acid and dichloromethane (1:1) (10 ml) for 10 min at room temperature. The reaction was monitored by TLC. After completion of the reaction, the reaction mixture was diluted with ethyl acetate, washed with aqueous NaHCO₃ followed by water and dried over Na₂SO₄. The residue obtained after removal of the solvent was chromatographed over a column of silica gel (230-400 mesh, using chloroform: acetone=92:8 as an eluent) to obtain 3-acetoxy andrographolide (100 mg) as a colorless solid, m.p. 205° C., m/z 392.

¹H-NMR(CDCl₃): δ6.95 (t, 1H, H-12), 5.0 (d, 1H, H-14), 4.9 (s, 1H, H-17a), 4.65 (m), 4.6 (s, 1H, H-17b), 4.45 (dd, 1H, H-3), 4.25 (d), 4.15 (d, 1H, H-19a), 3.4 (d, 1H, H-19b), 2.5 (m), 2.1 (s, 3H), 1.2-2.0 (m), 1.1 (s, 3H), 0.7 (s, 3H).

### REFERENCE

(1) Rec. Trav. Chim. 1911, 30, 151˜160.
(2) (a) Toxicology in Vitro. 1999, 13(6), 897˜903.
   (b) Fitoterpia. 1995, 66(5), 415˜420.
   (c) International Journal of Pharmacognosy. 1995, 33(2), 135˜138.
   (d) Asia Pacific Journal of Pharmacology. 1994, 9(2), 73˜78.
   (e) Journal of Ethnopharmacology. 1993, 40(2), 131˜136.
(3) (a) Zhongguo Zhongyao, 2002, 27(2), 147˜150.
   (b) Journal of Natural Products. 1993, 56(7), 995˜999.
(4) (a) Biological and Pharmaceutical Bulletin. 2002, 25(9), 1169˜1174.
   (b) British Journal of Pharmacology. 2002, 135(2), 399˜406.
   (c) Planta Medica. 2000, 66(4), 314˜317.
   (d) British Journal of Pharmacology. 2000, 129(8), 1553˜1560.
   (e) Phytotherapy Research. 1998, 12(l), 37˜40.
   (f) Fitoterpia. 1996, 67(5), 452˜458.
(5) (a) Life Science. 2000, 66(8), 709˜723.
   (b) Indian Journal of Pharmacology. 1994, 26(4), 296˜300.
(6) Journal of Experimental Therapeutics and Oncology. 2003, 3(3), 10147˜158.
(7) Phytotherapy Research. 2000, 14(5), 333˜338.
(8) (a) BMC Pharmacology. 2003, 3.
   (b) Drug Delivery. 2000, 7(4), 209˜213.
(9) Phytomedicine. 2002, 9(7), 589˜597.
(10) CN 1293955. CN 1266699.
(11) Phytomedicine. 1999, 6(1), 27˜31.
(12) Pharmacological Research. 1998, 38(6), 413˜417.
(13) Phytotherapy Research. 1999, 13(2), 157˜159.
(14) (a) Indian Journal of Pharmaceutical Sciences. 1998, 60(3), 176-178.
   (b) Pharmaceutical Biology. 1998, 36(1), 72˜74.
(15) International Journal of Crude Drug Research. 1990, 28(4), 273˜283.
(16) (a) Bangladesh Pharmaceutical Journal. 1977, 6(2), 21˜24.
   (b) Planta Medica. 2003, 69(12), 1075˜1079.
(17) WO 2003035007. WO 2003007975
(18) Phytotherapy Research. 1998, 12(1), 37˜40.

What is claimed is:

1. A compound of the formula (I) 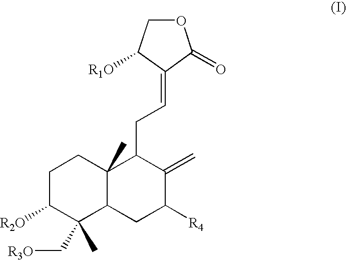
where
R¹, R² and R³ may be same or different and independently represent substituted or unsubstituted groups such as alkyl, aryl, heteroaryl, aralkyl, heteroaralkyl, alkanoyl, alkenoyl, aroyl, heteroaroyl, aralkanoyl, arakenoyl, heteroaralkanoyl, heteroaralkenoyl, sulfonyl group or a group —(CO)—W—R⁵, where W represents O, S or NR⁶, wherein R⁶ represents hydrogen or (C₁-C₆)alkyl group;
R⁵ represents substituted or unsubstituted groups such as alkyl, aryl, aralkyl, aroyl, OR², OR³ or a substituted or unsubstituted 6 or 7 membered cyclic structure containing carbon and oxygen atoms;
R⁴ represents halogen or XR⁷ where X represents O, S, NH and R⁷ represents hydrogen or substituted or unsubstituted groups such as alkyl, aryl, aralkyl, alkenoyl, alkanoyl, aroyl, heteroaroyl, aralkenoyl, aralkanoyl, sulfonyl groups or a group —(CO)—NH—R⁸ where R⁸ represents substituted or unsubstituted groups such as alkyl, aryl and aralkyl;
and the stereoisomers and pharmaceutically acceptable salts thereof.

2. A pharmaceutical composition for inhibiting or antagonizing TNFα in a mammal, including human, comprising an amount of andrographolide or a compound of formula (I) as defined in claim 1, or the stereoisomers or pharmaceutically acceptable salts thereof, that is effective in inhibiting or antagonizing TNFα, and a pharmaceutically acceptable carrier.

3. A pharmaceutical composition for treating diseases or conditions in which TNFα is involved, such as rheumatoid arthritis, Crohn's disease, plaque sclerosis, septic shock, cancer or cachexia associated with an immunodeficiency, comprising an amount of andrographolide or a compound of formula (I) as defined in claim 1, or the stereoisomers or pharmaceutically acceptable salts thereof, that is effective in inhibiting or antagonizing TNFα, and a pharmaceutically acceptable carrier.

4. A method of inhibiting or antagonizing TNFα in a mammal, including human, comprising administering to said mammal an amount of andrographolide or a compound of formula (I) as defined in claim 1, or the stereoisomers or pharmaceutically acceptable salts thereof, that is effective in inhibiting or antagonizing TNFα.

5. A method of treating diseases or conditions in which TNFα is involved, such as rheumatoid arthritis, Crohn's disease, plaque sclerosis, septic shock, cancer or cachexia associated with an immunodeficiency, comprising an amount of andrographolide or a compound of formula (I) as defined in claim 1, or the stereoisomers or pharmaceutically acceptable salts thereof, that is effective in treating such diseases or conditions.
